(19) United States
(12) Patent Application Publication
(10) Pub. No.: US 2008/0132580 A1
(43) Pub. Date: Jun. 5, 2008

(54) Dispersion For Delivering Active Agents

(76) Inventors: Sarveswara Rao Srirama Mandavilli, Hyderabad (IN); Venkat Arvind Vakati, Hyderabad (IN); Chithambaram Muthu Lingam, Tamil Nadu (IN); Govindan Saravana Perumal, Tamil Nadu (IN); Venkateswarlu Vobalaboina, Warangal (IN); Harshal Prabhakar Bhagwatwar, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/721,937

(22) PCT Filed: Dec. 16, 2005
(86) PCT No.: PCT/US05/45415

§ 371 Date: Jun. 15, 2007

Related U.S. Application Data
(60) Provisional application No. 60/637,193, filed on Dec. 17, 2004.

(51) Int. Cl. A61K 31/07

(52) U.S. Cl. 514/725

(57) ABSTRACT

A composition for topical application comprises a dispersed phase comprising an active agent and a polymer or lipid; and a gelled continuous phase comprising a viscosity modifier and a solvent. The composition can provide an active agent to skin or mucous membranes in a controlled release form.

### INTRODUCTION TO THE INVENTION

The present invention relates to the topical delivery of active agents.

In one aspect, the invention relates to a dispersion comprising a dispersed hydrophobic droplet phase and a continuous hydrophilic gel phase, which composition upon application to the body forms a film having a dispersed hydrophobic droplet phase and a continuous hydrophilic phase. By incorporating an active agent into the dispersion, a delivery composition is formed from which the active agent can be released in a desired fashion, such as over a prolonged duration. This invention also relates to processes by which the composition incorporating the active agent is made. The use of the delivery system and compositions for healthcare and cosmetic applications are described below.

Delivery of therapeutic agents for the treatment of disease is primarily through the oral and injectable routes. This is adequate for disease conditions which are systemic or spread out through the body or to areas in the body that the drug can reach through systemic administration. But these routes do not address disease conditions of areas of the body such as the skin, vagina, rectum, nose, eye, nails, and others where the disease is localized to these areas. Even though a fraction of the dose administered orally will reach these organs, a much larger fraction is distributed to the rest of the body resulting in non-target organ toxicity and incomplete efficacy, as the required dose does not reach the target areas.

Delivery of bioactive and bioinactive agents to and through the skin, nails, and mucous membranes is a field of drug delivery which is rapidly gaining importance. This is especially true for active agents which need to be delivered to the surface of the skin or through the skin into the different layers of the skin to treat a variety of disorders such as acne, dermatitis, psoriasis, leprosy, cancers of the skin, and the like. Similarly, delivery of the drug through a nail for the treatment of, for example, onychomycosis, poses a major problem for existing delivery compositions due to the hard and impermeable nature of the nail. Other forms of delivery such as topically, vaginally, rectally, nasally, and others, which require superficial application of a product for local therapy of diseases such as fungal and bacterial infections, local wounds, inflammation and others, are also of great interest as such therapy through local delivery would provide high local concentrations of the bioactive agents at the site of treatment. Further, for most such treatments the onset time for efficacy would be dramatically reduced as the drug would be immediately available as against oral administration where there is generally a lag time associated with the onset of action. Delivery compositions available for delivery of bioactive agents to these areas of the body include ointments, creams, gels, lotions, and foams, which are easy to administer and have excellent patient acceptability for short duration applications and for non-irritant compounds.

Most of these compositions release the active agent rapidly, resulting in the need for either repeated application, such as for antibiotics or in pain management, or are easily washed off, such as in sunscreen compositions or vaginal preparations, or further still result in extremely high irritation to the application site because of the irritant nature of the compounds, such as retinoids or benzoyl peroxide for the treatment of acne. To overcome these problems of irritancy and the requirement for frequent repeated applications, controlled release compositions have been developed.

Such compositions include a porous drug-loaded microbeads-in-a-gel delivery system which is marketed under the trade name RETIN-A MICRO™ for the delivery of tretinoin for the treatment of acne (see for example U.S. Pat. Nos. 5,955,109; 4,690,825; 5,135,740; and 5,316,774); the presence of microbeads results in an undesirable gritty feel of the product, when it is applied to skin. Other delivery compositions which attempt to overcome the above mentioned problems include incorporating microspheres or liposomes in gel delivery compositions, transdermal delivery systems and the like (U.S. Patent Application Publication No. 2002/0192243, and U.S. Pat. Nos. 4,624,665 and 6,468,552). These delivery compositions suffer from the drawbacks of use of strong volatile organic solvents in their manufacture, residual monomers and catalysts left over from the polymerization process, extremely high level of complexity in making the products, all leading to products which are difficult to make and scale-up resulting in higher costs to the patients.

International Application Publication W)2004/010988 and U.S. Pat. No. 6,211,250 both describe a homogeneous solution composition comprising a mixture of a hydrophilic and a hydrophobic polymer in a volatile organic solvent, further with an added bioactive agent, for application to a body. Upon application to a body, the organic solvent evaporates to form a dry film of the polymer mixture with a dispersed hydrophobic polymer phase and a continuous hydrophilic polymer phase, or vice versa, from which the drug is released in a controlled fashion. The composition uses ethanol, methanol, isopropanol, water, or their mixtures as volatile solvents. The products suffer from the drawbacks of the use of volatile organic solvents resulting in dry skin through the solubilization of skin lipids, formation of a solid film which would be formed poorly at rough edges such as around the contours of the face and elsewhere in the body, difficulty in washing off the film once it has been exhausted of the drug substance, and possible instability due to evaporation of the volatile solvents during manufacture and storage. Further, even though water has been described as one of the volatile solvents, the very nature of the composition makes it very difficult to formulate with only water as a volatile solvent. Also, the use of such a composition containing a volatile solvent in sensitive areas such as the vagina, rectum, nasal cavity, or open wounds on the skin such as burns and ulcers, psoriasis and leprosy; makes this a composition with very limited applicability.

European Patent Application 1 033 127 A1, U.S. Pat. No. 6,589,511 and International Application Publication WO 98/41190 describe a composition for forming microparticles in situ comprising an emulsion of a solution of a biodegradable polymer in an organic solvent in a continuous phase comprised of a polyhydric alcohol with an added viscosity enhancer and adhesive. The delivery system is designed and exemplified specifically for periodontal delivery, using biodegradable polymers only. The product is intended to form microparticles upon contact with an aqueous medium and is intended for periodontal use only.

An oil-in-water emulsion for topical application described in U.S. Pat. No. 5,618,522 comprises an oil phase thickened with particulate thickeners chosen from chemically treated or coated silica, polymethacrylate polymers, polymethacrylate and styrene copolymers, treated calcium silicate, treated bentonite, treated hectorite and their mixtures. The term “particulate thickener” means that the thickener is in the form of small, finely divided particles having a mean diameter of less than about 100 microns. The particulate thickener is essentially insoluble in the oil phase and is dispersed therein. The particulate thickener has a solubility of less than about 10%, preferably less than about 5%, and more preferably less than about 1% by weight of the oil phase, including the oil phase emulsifier, at 25° C.

A need remains for a delivery composition which is simple to prepare, economical when compared with prior compositions, easy to scale-up, non-irritating, does not use volatile and toxic organic solvents, and is capable of delivering single or multiple bioactive agents simultaneously to treat a wide range of disease conditions which are localized to diverse anatomical areas of the body such as skin, hair, scalp, vagina, rectum, nose, eye, ear and the like, and which can also be extended to oral administration.

### SUMMARY OF THE INVENTION

The present invention relates to a delivery system for the localized delivery of biologically active or bioinactive substances, and a ready-to-use, gelled dispersion composition for producing such a system, a process for preparing and administering the composition and a method of use for such a composition and system.

The delivery system is formed upon surface application of a gelled dispersion composition and takes the form of a film comprising droplets of a discontinuous hydrophobic phase in a continuous hydrophilic gelled phase.

In an aspect, the invention includes a composition for topical application comprising a dispersed phase comprising an active agent and a polymer or lipid; and a gelled continuous phase comprising a viscosity modifier and a solvent.

In another aspect, the gelled dispersion composition comprises:
(1) a solution, dispersion or gel of a hydrophobic, water-insoluble biocompatible polymer or a lipid (non-polymeric material) or mixtures thereof in a biocompatible, water-insoluble, non-volatile organic solvent(s), dispersed uniformly in
(2) a biocompatible hydrophilic continuous phase vehicle comprised of water or a polyhydric alcohol or mixtures thereof and a viscosity enhancer or gelling agent,
resulting in a stable, gelled, polymer/lipid (non-polymeric material) solution dispersion or gel-in-gel dispersion.

In a further aspect, the invention includes a process for preparing a composition for topical application, comprising:
combining components comprising an active agent and a polymer or lipid to form a dispersible phase;
combining components comprising a viscosity modifier and a solvent, to form a gel-forming composition; and
dispersing a dispersible phase in a gel-forming composition to form a dispersion having a gel continuous phase.

A still further aspect of the invention comprises a process for preparing a composition for topical application, comprising:
dissolving tretinoin in a dispersible composition comprising a polymer or an oil;
combining gel-forming composition components comprising water and a polymer; and
dispersing a dispersible composition in a gel-forming composition.

A dispersion of the invention upon application to a body forms a film with a dispersed hydrophobic droplet phase and a continuous hydrophilic phase, wherein each of the hydrophobic droplets of the dispersed phase functions as a distinct site for the release of bioactive or bioinactive materials.

The composition has excellent spreadability and possesses the characteristics of rapidly forming the delivery system upon application to a body surface. When a biologically active agent is added to the delivery composition a drug delivery composition is formed. In this situation, the drug will begin to be released as soon as the composition is applied to the body, for example to the skin.

In another embodiment of the invention, other ingredients such as sunscreens, emollients, moisturizers and the like are added for a protective or cosmetic effect.

A feature of the delivery system of the invention is that the film formed from the composition does not dry rapidly as happens with the prior art delivery compositions utilizing volatile organic solvents (such as for example in WO 2004/010988 and U.S. Pat. No. 6,211,250). The delivery system thus formed has excellent retentiveness at the site of application and does not flake off. Further, a continuous phase comprising water or ingredients such as a polyhydric alcohol, or mixtures thereof, provides a smooth emollient feel to the skin.

A further aspect of the inventive delivery composition is that the films formed are flexible and thus can be applied to areas of a body such as joints and the like where there are discontinuities which would cause rupture of films formed from prior delivery compositions. An absence of volatile organic solvents such as ethanol and the like also leads to a composition which is stable during a prolonged shelf-life with no change in the contents such as would occur due to evaporation of alcohol and other volatile organic solvents. The absence of volatile organic solvents also results in a reduced toxicity to the skin. A further important aspect of the delivery composition is the ease with which the delivery composition can be washed off the skin when compared with prior art film-forming compositions based on volatile solvents. This also results in a reduced irritation to the skin.

The bioactive agent incorporated into the delivery composition is protected from the atmosphere, leading to an enhanced chemical stability of unstable bioactives. Further, the bioactive agent does not come in contact with the skin directly in large amounts/concentrations as with the prior art delivery compositions and is delivered slowly from the dispersed phase droplets through the continuous phase. This can provide a controlled release of the active agent resulting in a reduced number of applications of the product and also a reduction in the irritancy potential of irritant compounds.

The dispersed phase is of a controlled droplet size distribution with droplets ranging in size from 0.1-400 μm, or about 1-150 μm. The processing conditions such as where applicable, the speed of homogenization, and the molecular structure of the final gel will determine the size, distribution and shape of the droplets. These characteristics are maintained by the viscous gelled nature of the dispersion.

Another important characteristic of the dispersed hydrophobic phases is their semisolid to gelled consistency, in contrast to the solid microparticles dispersed in a continuous gel phase (as in U.S. Pat. Nos. 4,690,825; 5,955,109; 5,135,740; 5,316,774) or a solid hydrophobic phase formed from the evaporation of a homogeneous solution of a mixture of hydrophobic and hydrophilic polymers in a volatile organic solvents (as in WO 2004/010988 and U.S. Pat. No. 6,211,250). There may also be a small percentage of solid suspended particles present in the dispersed phase depending upon the particular solvent-polymer/lipid composition chosen.

### DETAILED DESCRIPTION

The terms “active agent,” “therapeutic agent,” “bioactive agent,” and “biologically active agent” are used interchangeably and as used herein are intended to denote substances that have a physiologic effect, for example, a drug.

The term “bioinactive agent” as used herein is intended to mean a compound which has an adjunct effect such as an emollient, moisturizer, wrinkle remover, antioxidant, or other material used in cosmetic compositions for enhancing the appearance characteristics of humans.

The term “film forming” as used herein is intended to mean a substance capable of forming a thin layer on the surface to which it is applied, when exposed to ambient conditions.

The term “percutaneous” as used herein is intended to mean any route of administering an active agent onto, into or through the skin of a subject so as to achieve one or more of a topical, local or systemic physiologic effect.

Similarly, the term “perungual” as used herein is intended to mean any route of administering an active agent onto, into or through the nail of a subject so as to achieve one or more of a topical, local or systemic physiological effect.

In an embodiment, a dispersion composition of the present invention comprises:

1. a solution, dispersion or gel of a hydrophobic, water-insoluble biocompatible polymer or a lipid (non-polymeric material) or mixtures thereof in a biocompatible, water-insoluble, non-volatile organic solvent(s), dispersed uniformly in

2. a biocompatible hydrophilic continuous phase vehicle comprised of water or a polyhydric alcohol or mixtures thereof and a viscosity enhancer or gelling agent,

resulting in a stable, gelled, polymer/lipid (non-polymeric material) solution dispersion or gel-in-gel dispersion.

### Biocompatible Polymers of Lipids (Non-Polymeric Materials)

### Polymers

The polymeric materials useful in the present compositions are those that are biocompatible, substantially insoluble in water and body fluids, and are capable of being at least partially solubilized in the organic solvents of the invention.

The polymer is a long chain polymer, amorphous, semicrystalline, or crystalline in nature, and can be from a natural, synthetic or semisynthetic source. Preferably, the long chain polymer is one with a molecular weight in the range of 500 to 10,000,000 daltons as measured by gel permeation chromatography against polystyrene standards. Examples of such polymers useful in this invention include, but are not limited to, ethyl celluloses, acrylates, methacrylates, pyrrolidones, polyoxyethylenes, hydroxypropylmethyl celluloses, hydroxypropyl celluloses, polymethylmethacrylates, cellulose acetates and their derivatives, cellulose acetate phthalate, hydroxypropyl methylcellulose phthalate, shellac, methacrylic acid based polymers such as those sold under the tradename EUDRAGIT™, zein, polycarbonates, polyorthoesters, polydioxanones, polyacetals, polyhydroxybutyrates, polyhydroxyvalerates, polyethers, polyphosphazenes, polyhydroxycelluloses, polyalkylene oxalates, polyorthocarbonates, polyphosphoesters, star-branched polymers and copolymers, polysaccharides, polyketals, polyalkylene succinates, polypropylene oxides, chitin, chitosan, and other polymers known to a person skilled in the art of drug delivery, including copolymers, terpolymers, combinations, and mixtures thereof.

These polymers can either be used alone or as copolymers created from different monomers in different ratios or mixtures of two or more different polymers or copolymers to achieve a variety of release profiles. The copolymers could either be random copolymers, in a variety of co-monomer ratios, or block copolymers. Water-soluble polymers such as polyethylene glycol, polyvinyl alcohol, polyvinylpyrrolidone, polyoxyethylene-polypropylene block copolymers or other water-soluble polymers can be copolymerized with any of the polymers that can be used in this invention to modify their properties. Additionally, these polymers can also be copolymerized with other monomers not specifically mentioned here. Polymers with different substituent groups grafted onto a polymer backbone or polymers or copolymers with different or modified end groups such as to provide a charge or to enhance the permeability characteristics or to modify the lipophilicity-hydrophilicity balance of the polymer are within the scope of this invention.

In one embodiment of this invention, a mixture of polymers is used in the preparation of the composition. Other polymers which are water-soluble or water-gellable such as gelatin, collagen, albumin, fibrin, fibrinogen, beta-cyclodextrin, polyoxyethylene-polypropylene block copolymers, polyethylene glycols, polyethylene oxides, polyvinyl alcohol, polyvinyl pyrrolidone, methyl celluloses, and others can also be used in the invention in combination with any of the water-insoluble polymers described above.

The use of polymer blends allows the formation of polymers of different hydrophilic-hydrophobic characteristics with simple mixing, and without substantially changing a polymer. Thus, polymers of two or more kinds can be simply blended and used in the preparation of the delivery system of the invention. The polymers can be mixed in any ratio from 100:0 to 0:100 percent w/w. The kinds of polymers to be blended, the actual percentages of the polymers and the ratios in which they are to be mixed will be readily apparent to a person skilled in the art of preparing polymeric drug delivery systems. For example, if a polymer mixture with greater hydrophilicity is required, then a water-insoluble and water-soluble polymer are mixed and a higher percentage of the water-soluble polymer is used. If a more hydrophobic polymer mixture is required, then a higher percentage of the water-insoluble polymer is used. It can also be beneficial to mix two polymers with differing characteristics such as for example a mixture of two viscosity grades of the same polymer resulting in a final solution with a different viscosity than is possible by the use of either polymer alone, or a mixture of polymers with different degrees of substitution or crosslinking, or with different degrees of crystallinity. Such physicochemical characteristics of different polymers can be utilized together to achieve a wide variety of release characteristics for a wide variety of bioactive and bioinactive agents.

Other classes of polymers which are not described here but are known to those skilled in the art also fall within the scope of this invention. There is no limitation to the polymers chosen for use in the composition as long as the polymers are at least partially soluble in the solvents of the invention and serve to create a hydrophobic dispersed phase.

### Lipids (Non-Polymeric Materials)

Non-polymeric materials useful in the present compositions are those that are biocompatible, substantially insoluble in water and body fluids, and are capable of being at least partially solubilized in the organic solvents of the invention.

Non-polymeric materials that can be used in the composition generally include any having the foregoing characteristics. Examples of useful non-polymeric materials include, without limitation thereto: sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and tri-acylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate, glyceryl tribehenate, and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; waxes such as carnauba wax, beeswax, paraffin wax, microcrystalline wax, spermaceti, hydrogenated vegetable oils, and their purified forms and derivatives; phospholipids such as phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols; and combinations and mixtures thereof. These materials can either be used alone or in combination with one or more other material including the polymeric materials described above.

In another embodiment of the invention, copolymers or graft polymers of one of the polymers described above and a lipid (non-polymeric material) or a phospholipid or a lipoprotein or a emulsifier can be used as the hydrophobic material in the dispersed droplet phase. Further, any of the materials described above can be conjugated with a biologically active agent to form a prodrug and then be incorporated into the composition. The use of such a prodrug approach is well within the scope of this invention.

There is no limitation on the kind of polymer or non-polymeric material that can be chosen as long as it is at least partially soluble in the solvent systems of this invention at the temperature of processing and that of storage and has the capability to enhance the viscosity of the hydrophobic dispersed droplet phase.

### Biocompatible Organic Solvents

The solvents of the invention should be biocompatible, non-volatile and water-immiscible. The term “water-immiscible” is intended to mean solvents which have a solubility of about less than about 10-20% by weight in aqueous media. More specifically, the term applies to the substantial immiscibility of the solvent in the hydrophilic continuous phase of the invention. Thus, the solvent is chosen so that it is capable of dissolving (at least partially) the polymeric or non-polymeric hydrophobic material at expected temperatures of processing and storage, dissolve or suspend the biologically active agent and also be immiscible with the continuous phase, so that upon mixing a dispersed hydrophobic phase can be formed within a continuous hydrophilic phase.

The term “aqueous media” is intended to include water, buffer solutions to provide different pH values, and various body fluids.

Miscibility in the continuous phase media can be determined experimentally as follows: media (such as 1-5 g) is weighed into a clear container at a controlled temperature, such as about 20° C., and a candidate solvent is added dropwise. The solution is swirled to observe phase separation. When the saturation point appears to be reached, as determined by observation of phase separation, the solution is allowed to stand overnight and is re-checked the following day. If the solution is still saturated, as determined by observation of phase separation, then the percentage (w/w) of solvent added is determined. Otherwise, more solvent is added and the process is repeated. Solubility or miscibility is determined by dividing the total weight of solvent added by the final weight of the solvent/water mixture. When solvent mixtures are used, for example 20% triacetin and 80% benzyl benzoate, they are pre-mixed prior to adding to the water.

Suitable solvents are exemplified by, but not limited to, triacetin, benzyl alcohol, benzyl benzoate, dibutyl sebacate, ethyl oleate, isopropyl myristate, the lower alkyl and aralkyl esters of aryl acids such as benzoic acid, the phthalic acids, salicylic acid, lower alkyl esters of citric acid, such as triethyl citrate and tributyl citrate and the like, and aryl, aralkyl and lower alkyl ketones.

Art recognized benzoic acid derivatives from which solvents having the requisite solubility can be chosen include, without limitation, 1,4-cyclohexane dimethanol dibenzoate, diethylene glycol dibenzoate, dipropylene glycol dibenzoate, polypropylene glycol dibenzoate, propylene glycol dibenzoate, diethylene glycol benzoate and dipropylene glycol benzoate blend, polyethylene glycol (200) dibenzoate, isodecyl benzoate, neopentyl glycol dibenzoate, glyceryl tribenzoate, pentaerylthritol tetrabenzoate, cumylphenyl benzoate, and trimethyl pentanediol dibenzoate.

Art recognized phthalic acid derivatives from which solvents having the requisite solubility can be chosen include, without limitation, alkyl benzyl phthalate, bis-cumyl-phenyl isophthalate, dibutoxyethyl phthalate, dimethyl phthalate, dimethyl phthalate, diethyl phthalate, dibutyl phthalate, diisobutyl phthalate, butyl octyl phthalate, diisoheptyl phthalate, butyl octyl phthalate, diisonoyl phthalate, nonyl undecyl phthalate, dioctyl phthalate, di-iso octyl phthalate, dicapryl phthalate, mixed alcohol phthalate, di-(2-ethylhexyl) phthalate, linear heptyl, nonyl, phthalate, linear heptyl, nonyl, undecyl phthalate, linear nonyl phthalate, linear nonyl undecyl phthalate, linear dinoyl, didecyl phthalate (diisodecyl phthalate), diundecyl phthalate, ditridecyl phthalate, undecyldodecyl phthalate, decyltridecyl phthalate, blends (such as 50/50) of dioctyl and didecyl phthalates, butyl benzyl phthalate, and dicyclohexyl phthalate.

Oils can also be used as solvents when they solubilize certain polymeric or non-polymeric materials such as waxes, lipids, aluminum monostearate, sorbitan monostearate, sorbitan monopalmitate, to name a few. Useful oils include oils from natural, synthetic or semisynthetic origin. Such oils include, without limitation, various grades of animal oils such as whale oil or shark liver oil, or vegetable oils such as sesame seed oil, cottonseed oil, poppy seed oil, castor oil, coconut oil, canola oil, sunflower seed oil, peanut oil, corn oil, soybean oil, or their fractionated counterparts such as capric-caprylic triglycerides and their salts with other acids. Super refined varieties such as those available from Croda International plc, of Goole, East Yorkshire, United Kingdom, paraffin oil, silicone oil, and other oils useful in the area of pharmaceutical drug delivery can be used in the invention.

In one specific embodiment of the invention, the hydrophobic agent is sorbitan monostearate or sorbitan monopalmitate, either of which dissolves in a number of solvents or oils at elevated temperatures, and then gels the solvent or oil as the temperature is lowered to room temperature. Such gelled solvent droplets when dispersed within a continuous hydrophilic phase are also included within the scope of this invention.

In a further embodiment of the invention, the hydrophilicity of the solvent can be modified by mixing the water-insoluble solvents as described above with solvents which are water-miscible. Such solvents include N,N′-dimethylacetamide (DMA), glycofural, dimethylsulfoxide (DMSO), N-methyl-2-pyrrolidone (NMP), water, 2-pyrrolidone, ethanol, propylene glycol, polyethylene glycol, glycerol, sorbitol, dimethylformamide (DMF), dimethyl acetamide, dialkylamides, caprolactam, glycerolformal, decylmethyl sulfoxide, and other polar solvents, because of their non-volatility and their miscibility with water and with each other. Such mixing allows the preparation of compositions where the polymer/non-polymeric material is completely soluble in the solvent system as against only a partial solubility in the water-immiscible solvent alone, allowing a wide modification in the release of the active agent from the hydrophobic droplet phase.

The viscosity of the polymer/non-polymeric material solution is governed by the solvent system used for solubilization, the type of polymer/non-polymeric material, concentration of the polymer/non-polymeric material and molecular weight of the polymer/non-polymeric material, and whether the polymer/non-polymeric material is completely in solution or is present in a partially solubilized state. A particular solvent or solvent composition (combinations) should be chosen for each polymer/non-polymeric material to provide a polymer/non-polymeric material solution of optimum solubility and of optimum viscosity. In any event, to get an optimum product it is preferable to use a polymer/non-polymeric material and solvent combination where the polymer/non-polymeric material will form a clear solution at the temperature of processing. It is quite possible that some of the polymer/nonpolymeric material may precipitate within the hydrophobic phase droplet after the dispersion has been formulated and brought to room temperature. Such compositions are also within the scope of the invention.

When a drug will be incorporated into the polymer/non-polymeric material solution, the solvent used in the invention should provide a polymer/non-polymeric material solution/dispersion with a high enough viscosity to carry a fairly high drug load but should not be too viscous for processing for the purposes of the invention. This is also true when a bioinactive agent is used. The choice of solvents and solvent systems for different polymer/non-polymeric material is within the scope of understanding for a person skilled in the art of making polymer/non-polymeric material-based drug delivery systems.

### Polymer Concentrations

As described above, the polymer/non-polymeric material could be completely in solution or be partially in solution and be held partially as a suspension. A polymer/non-polymeric material when incorporated into the solvent system of the invention can form a clear solution at the elevated temperature required for processing, but precipitate partially when cooled to lower temperatures. As long as the polymer/non-polymeric material is partially in solution at room temperature, the novel dispersions of the invention can be made and will provide the delivery of the active agent.

Generally, concentrations of polymer/non-polymeric material will be about 1-90% w/w with respect to the solvent in the hydrophobic dispersed phase, or about 5-70% w/w, or about 10-60% w/w. A frequently optimum concentration is about 10-60% w/w with respect to the solvent. The molecular weight of the polymer, copolymer or mixtures of polymers and their crystallinity frequently will determine the solution viscosity. Thus a higher molecular weight polymer will frequently provide a solution of higher viscosity at a lower concentration when compared with a lower molecular weight polymer from the same class. Polymer/non-polymeric material solutions of concentrations of up to 60% w/w can be processed by raising the temperature of the polymer solution, such as up to 25-90° C. Such concentrated polymer/non-polymeric material solutions allow the delivery of higher loads of biologically active substances in smaller volumes of the final delivery system.

The polymer/non-polymeric material solution will generally comprise about 0.01-60% w/w of the total composition, or about 5-50% w/w, or about 10-40% w/w.

### The Hydrophilic Continuous Phase

The hydrophilic continuous phase should be biocompatible and should not cause any undue irritation to the application site. It should have the capacity to incorporate and hold within its structure, large amounts of the dispersed phase without causing phase separation at the temperature of storage. Further, the continuous phase should be immiscible with the dispersed hydrophobic droplet phase and have excellent spreadability on application to a surface to rapidly from a film and thus the delivery system of the composition.

Exemplary hydrophilic vehicles include, without limitation, water, glycerol, propylene glycol, sorbitol and other higher alcohols, and their mixtures in varying proportions. A small percentage of a volatile solvent such as ethanol, acetone, ethyl acetate, and the like can be incorporated to aid processing and is within the scope of the invention.

The viscosity of the continuous phase is enhanced through the addition of hydrophilic viscosity enhancers or gelling agents such as carbopols, carboxyvinyl polymers, alginic acid and its salts, gelatin, polyvinyl alcohol, high molecular weight polyethylene glycols, hydroxypropyl cellulose, hydroxypropyl methylcellulose, methylcellulose, sodium carboxymethylcellulose, and other materials used for enhancing the viscosity of liquids.

Appropriate agents can be added to achieve desired pH, salt concentrations, colors, and fragrances. In addition, any desired preservatives, antioxidants, opacifiers, emulsifiers, surfactants, emollients; permeation enhancers can be added to improve the performance and esthetic properties of the delivery composition. The use of such materials for formulation of an esthetically appealing and stable composition for local application is well known to a person skilled in the art of preparing pharmaceutical products. Different classes of compounds from all of these categories are all included within the scope of this invention without limitation.

The continuous hydrophilic phase can comprise about 30-95% w/w of the total composition, or about 35-80% w/w of the total composition.

### Process of Manufacture of the Composition

In general, a dispersible phase, containing an active agent, is dispersed in a gel to form the compositions of the invention.

A representative process of preparation for the dispersion of the invention comprises the steps of:
(a) dissolving a biocompatible polymer/non-polymeric material in a biocompatible water-insoluble, non-volatile organic solvent or a mixture of solvents, if necessary at an elevated temperature, to form a hydrophobic solution;
(b) separately preparing a continuous hydrophilic phase with the addition of the viscosity enhancer in water or a mixture of water with a water-miscible solvent, if necessary at an elevated temperature;
(c) emulsifying the hydrophobic solution as described in (a) above into the continuous hydrophilic phase as described in (b) above to form a dispersion, and
(d) mixing the dispersion and subsequently cooling it while mixing continuously, to obtain the final gelled dispersion.

The hydrophobic solution at an elevated temperature (such as about 65-100° C.) is dispersed in the continuous phase at the same temperature, preferably, in a flow-through cell or a static mixer and with the aid of shear provided by high-speed homogenization, such as at speeds of 500-25,000 rpm, probe sonication, high pressure homogenization, atomization through a spray nozzle under pressure of a compressed gas, or atomization through an ultrasonic nozzle. In some instances, the temperatures of the oil and polymer phases can be about 65-100° C., depending upon the stability of the excipients, the polymer in the solvent and if present, the biologically active or bioinactive agent. Where a polymer/non-polymeric material solution can be formed in the desired concentration at room temperature, the entire processing can be carried out at room temperature.

Addition of the gelling agent to the continuous phase after formation of the dispersion is also possible. So also, dispersion of a material such as a carbopol in the hydrophilic phase, followed by its neutralization after the formation of the dispersion of the hydrophobic phase in the continuous phase, is within the scope of the invention. Other methods of preparation of such dispersions are also included in this invention as long as a hydrophobic dispersed phase and a hydrophilic continuous phase exist together in the form of a dispersion as described above. The dispersion can be cooled either through continuous mixing while cooling to ambient temperatures of about 0-30° C. or by placing the dispersion in a low temperature environment, such as at about −20° C.

It is also possible to manufacture the dispersions at an elevated temperature (65-100° C.) and subsequently cool by refrigeration to low temperatures, such as about 2-8° C., with continuous homogenization to achieve a product having enhanced uniformity. The homogenization speed during this cooling step can be the same as that in the dispersion step or can be changed to a higher or lower speed. Frequently, the homogenization speed will be higher during the dispersion step and lower during the cooling step. It is also sometimes useful to re-homogenize the dispersions once they have been brought to refrigeration temperatures. The exact homogenization speeds to be used and the time for which homogenization should be carried out can be readily determined by a person skilled in the art of manufacturing disperse systems.

It is of course understood that the manufacturing process as described above could be readily extended to other forms of shear apart from high speed homogenization, such as high pressure homogenization, microfluidization, passing through a colloid mill or triple roller mill, and such other methods of providing shear as are known in the art of manufacture of dispersed systems. It is also possible to use a combination of the above mentioned procedures of providing shear. For example, a gelled composition can be prepared using high-speed homogenization as described above. This gelled composition can be used as a feed material for further high-pressure homogenization or micro fluidization to further reduce the droplet size as desired. The various parameters including homogenization pressure, number of cycles, processing temperature and such other parameters which govern the efficiency of high-pressure homogenization or micro fluidization will then govern the final outcome. Whatever the method or combination of methods used, the final outcome will be a gelled dispersion capable of forming the delivery system of the invention.

The droplet size of the dispersion will usually influence the rate of drug release. Typically, the smaller the droplet size, the greater the surface area and hence the faster the rate of release upon formation of the delivery system after application. A droplet size of about 0.1-400 μm, or about 5-150 μm, with greater than 40-60% of the droplets having an average size less than about 100 μm, generally is desirable. The size can be varied by a person skilled in the art of manufacture of dispersions by the variation in the sizes of the homogenizer probes used, the speed of homogenization, the temperatures of both the phases, the polymer concentration in the organic solvent, the ratio of the discontinuous (hydrophobic phase) to continuous (hydrophilic phase) phases, and such other parameters apparent to the person skilled in the art of disperse systems and drug delivery, and are all included herein.

### The Biologically Active Agent

The terms “drug,” “bioactive agent,” and “biologically active agent” as used herein include, without limitation, physiologically or pharmacologically active substances that act locally or systemically in a body. These terms are used interchangeably in the specification and claims. A “body” includes, but is not limited to, a human body or an animal body. Representative drugs and biologically active agents that can be used with the dispersions of the invention include, without limitation, peptide drugs, protein drugs, desensitizing agents, antigens, vaccines, anti-infectives, antibiotics, antifungals, antiacne, vitamins and analogs, retinoids, antileprotic, antimicrobials, antineoplastics, antitumor, antiallergenics, steroidal anti-inflammatory agents, anesthetics, decongestants, miotics, anticholinergics, sympathomimetics, sedatives, hypnotics, antipsychotics, psychic energizers, tranquilizers, androgenic steroids, estrogens, progestational agents, humoral agents, prostaglandins, analgesics, antispasmodics, antimalarials, antihistamines, cardioactive agents, non-steroidal anti-inflammatory agents, antiparkinsonian agents, antihypertensive agents, beta-adrenergic blocking agents, nutritional agents, antivirals, DNA fragments, nucleic acids, genetic material, oligonucleotides, radioisotopes, or any combinations of these classes of compounds. To those skilled in the art, other drugs or biologically active agents that can be applied to a body can be utilized in the described delivery system. Also, various forms of the drugs or biologically active agents may be used. These include, without limitation, forms such as uncharged molecules, molecular complexes, salts, ethers, esters, amides, and other chemically modified forms of the biologically active agent which are biologically active in or on a body.

Useful bioactive agents include, but are not limited to: sunscreens such as octyl methoxycinnamate, oxybenzone and avobenzone; anti-wrinkling/anti-aging agents such as retinoids (for example, retinoic acid, retinol, retinal, retinyl acetate retinyl palmitate, tretinoin, isotretinoin, adapalene, tazarotene and azelaic acid); alpha hydroxy acids; galactose sugars; antifungal agents like miconazole, econazole, ketoconazole, itraconazole, fluconazole, bifoconazole, terconazole, saperconazole, butoconazole, tioconazole, oxiconazole, sulconazole, clotrimazole, undecylenic acid, haloprogin, butenafine, tolnaftate, nystatin, ciclopirox olamine, terbinafine, amorolfine, naftifine, elubiol, griseofulvin; antibiotic and antiseptic agents such as mupirocin, neomycin sulfate, bacitracin, polymyxin B, ofloxacin, tetracyclines (chlortetracycline hydrochloride and oxytetracycline hydrochloride), clindamycin phosphate, gentamicin sulfate, benzalkonium chloride, benzethonium chloride, hexylresorcinol, methylbenzethonium chloride, phenol, quaternary ammonium compounds, triclocarbon, triclosan, tea tree oil and benzoyl peroxide; anti-acne and antipsoriatic agents such as corticosteroids (like betamethasone dipropionate, betamethasone valerate, clobetasol propionate, diflorasone diacetate, halobetasol propionate, amcinonide, desoximetasone, fluocinonide, fluocinolone acetonide, halcinonide, triamcinolone acetate, hydrocortisone, hydrocortisone valerate, hydrocortisone butyrate, aclometasone dipropionte, flurandrenolide, mometasone furoate and methylprednisolone acetate), vitamin D and its analogues; depigmentating agents; skin lightening agents (for example, kojic acid, ascorbic acid and derivatives like ascorbyl pamiltate); anti-oxidants such as tocopherol and esters, sodium metabisulfite, N-acetyl-cysteine, acetyl-cysteine, lipoic acid and dihydrolipoic acid, resveratrol, lactoferin, ascorbic acid, and derivatives thereof; metal chelators; sun blockers such as titanium oxide and zinc oxide; and skin protectants; including their pharmaceutically acceptable salts, esters, hydrates, solvates, etc., and mixtures of bioactive agents.

### The Biologically Inactive Agent

The term “biologically inactive agent” as defined for purposes of this invention includes without limitation compounds and components such as humectants, emollients, sunscreens, exfoliants such as lactic acid, glycolic acid, or other hydroxy acids, glycerol, perfumes, antioxidants, and other compounds and compositions that are useful in the preparation of compositions for protective, cosmetic, and other functions. The terms “biologically inactive agent” and “bioinactive agent” are used interchangeably in the specification and claims.

Certain materials can function as cosmetic ingredients, but also have some therapeutic uses, so would be categorized as both bioactive and bioinactive. Regulatory authorities can differ in their views regarding some ingredients: sunscreening products are regarded as nonprescription drugs in some countries, and as being cosmetic products in other countries, further lessening the distinction between bioactive and bioinactive agents. For these reasons, the term “active agent” is considered to encompass bioactive and bioinactive ingredients that have one or more of biologic, protective, and cosmetic functions.

### The Drug Delivery System

An envisioned use of the novel gelled dispersion is to provide a drug-delivery system. Accordingly, in one embodiment, a bioactive agent is added to the hydrophobic solution prior to emulsification. The drug can also be added as a solution or suspension. The drug in the hydrophobic phase can be from 0.01-50% w/w with respect to the polymer/non-polymeric material in the hydrophobic phase. In some cases, the drug will also be soluble in the solvent, and a homogenous solution of polymer and drug will be available. In other cases, the drug will not be soluble in the solvent, and a suspension, emulsion, or dispersion of the drug in the polymer solution will result. This suspension or dispersion can also be subjected to emulsification. In either case, upon application of the novel dispersions to a body, a film will be formed rapidly containing a dispersed hydrophobic droplet phase and a continuous hydrophilic phase from which the drug will be released.

In order to provide an initial release of biologically active agent where required, the drug can also be added directly into the continuous phase either as a solution (where the drug is soluble) or as a suspension (where the drug is insoluble).

The biologically active agent can be added to the hydrophobic phase and/or the continuous hydrophilic phase, either as a solution or a suspension depending upon the solubilities of the drug in the two phases. Either way, the formation of the delivery system from the composition and controlled release of the biologically active agent will follow.

The amount of drug or biologically active agent incorporated into the dispersion depends upon the desired release profile, the concentration of drug required for a biological effect, and the length of time that the drug has to be released for treatment. There is no critical upper limit on the amount of drug incorporated into the polymer solution or the continuous phase except for that of an acceptable solution or dispersion viscosity for processing and application. The lower limit of drug incorporated into the delivery system is dependent simply upon the activity of the drug and the length of time needed for treatment.

The release of drug from the delivery system can be affected by the dispersed phase concentration and the drug content therein, the hydrophilicity and viscosity of the continuous phase, the size and shape of the droplets, the loading of drug, the permeability factors involving the drug and the particular polymer/non-polymeric material, and other factors. Depending upon the drug selected for delivery, the above parameters can be adjusted by one skilled in the art of drug delivery to give the desired rate and duration of release. The continuous phase can itself behave as a controlled release component because of the presence of the viscosity enhancing agent. Due to the controlled release properties of the composition, drug release from a formed film to the underlying tissue is prolonged, such as extending over a period of several hours. For example, drug can be released over a period of 2 hours, or 4 hours, or 6 hours, or 8 hours, or over other time periods.

Bioinactive agents can be used with or in lieu of the drug, bioactive agent or biologically active agent in the drug delivery system in the same way as described above.

In one aspect of the invention, more than one biologically active agent can be delivered from a single composition. This can be achieved through having different actives loaded into the dispersed and continuous phase, or having two or more different species of dispersed phase droplets within the same hydrophilic continuous phase. In yet another aspect of the invention, such delivery of multiple agents can be achieved through the incorporation of preformed delivery vehicles into the novel gelled dispersion. Such preformed vehicles include for example liposomes, niosomes, micelles, microspheres, microcapsules, pellets, microparticles, and the like, being well known in the art of drug delivery. More than one species of particles can also be included. Further, more than one species of such dispersed phase droplets or particles made from different polymeric/non-polymeric materials can be used in concert to modulate the release of active agents.

### Mode of Administration

The dispersion of this invention can be part of a kit or device and be filled into tubes, jars, bottles, syringes, and any other form of packaging which will allow ease of application. The product will find use for the treatment of any condition which is localized to the skin, vagina, rectum, mouth, hair, scalp, nose, and any other such superficial location. In addition, the compositions can be used to apply bioactive substances that will be absorbed into the skin or mucous membranes, and thereby are introduced into the circulatory system to provide a systemic effect. The product can also be prepared as a sterile gel and provided in a sealed tube, bottle, etc. for use on open wounds, fractures, or burns. Where the compositions are to be administered rectally or vaginally, it is preferable to administer the compositions using appropriate applicators. Upon administration, release of the biologically active agent or bioinactive agent incorporated into the composition commences.

The dispersions can also be used in other fields such as agriculture, controlled release of pesticides, in aquaculture, veterinary drug delivery and other fields. Whatever may be the route of administration and whatever may be the field of application the general principles of formation of the delivery system will hold.

The following examples will further exemplify specific aspects and embodiments of the invention, and are not to be construed as limiting the scope of the invention, which is defined solely by the appended claims.

### EXAMPLE 1

### Dispersion Prepared Using Glyceryl Monostearate in the Hydrophobic Phase

| Component | Amount/100 g |
|---|---|
| Glyceryl monostearate | 3 |
| Triacetin | 10 |
| Carbomer* | 1 |
| Tromethamine solution (20% w/w in water) | 0.85 |
| Water (saturated with benzyl alcohol) | 85.05 |

*Carbopol ® Ultrez 10 Polymer, a crosslinked polyacrylic acid polymer from Noveon Inc., Cleveland, Ohio U.S.A.

Manufacturing process:

1. Glyceryl monostearate was dissolved in triacetin at a temperature of 70-80° C. to form the hydrophobic phase.

2. Carbomer was dispersed in the water under stirring at room temperature to form the hydrophilic phase.

3. The hydrophobic phase of step 1 was mixed with the hydrophilic phase of step 2 accompanied by homogenization at room temperature.

4. Tromethamine was added to the mixture of step 3 with continued homogenization at room temperature to form a gel.

The dispersion thus obtained was smooth when applied to the forearm of a volunteer and did not provide any gritty feeling thereby demonstrating the absence of any particulate matter.

The dispersion composition formed a smooth film with a discontinuous hydrophobic phase and a continuous hydrophilic phase.

### EXAMPLE 2

### Dispersion Comprising Benzyl Alcohol, Ethyl Cellulose, and Carbopol® Ultrez 10 Polymer

| Component | Amount/100 g |
|---|---|
| Ethyl cellulose 10 cps* | 1.2 |
| Benzyl alcohol | 6 |
| Carbomer | 1 |
| Tromethamine solution (20% w/w in water) | 1 |
| Water (saturated with benzyl alcohol) | 90.1 |

*Low viscosity ethyl cellulose (10 cps) sold under the brand name ETHOCEL by Colorcon (India) Ltd. was used in this and other examples.

The dispersion was prepared according to the process described in Example 1 except that in step 1 ethyl cellulose was dissolved in benzyl alcohol and all the steps were performed at room temperature.

### EXAMPLE 3

### Dispersion Comprising Benzyl Alcohol, Ethyl Cellulose, and Sodium Carboxymethyl Cellulose

| Component | Amount/100 g |
|---|---|
| Ethyl cellulose 10 cps | 1.2 |
| Benzyl alcohol | 6 |
| BLANOSE ® cellulose gum* | 2 |
| Water (saturated with benzyl | 90.1 |
| alcohol) | |

*BLANOSE ® cellulose gum-type 7H3SF is purified sodium carboxymethyl cellulose; 2% Brookfield viscosity is 20 mPas.

Manufacturing process:

1. Ethyl cellulose was dissolved in benzyl alcohol at room temperature to form the hydrophobic phase.

2. Sodium carboxymethyl cellulose was dispersed in the water under stirring at room temperature to form the hydrophilic phase.

3. The hydrophobic phase of step I was mixed with the hydrophilic phase of step 2 to form a gel.

### EXAMPLE 4

### Tretinoin Composition with Eudragit RS100

| | |
|---|---|
| Component | Amount/100 g |

Oil phase

| | |
|---|---|
| Tretinoin | 0.1 |
| EUDRAGIT RS100* | 1.2 |
| Benzyl alcohol | 6 |
| Butylated hydroxytoluene | 0.1 |

Aqueous phase

| | |
|---|---|
| Sodium lauryl sulphate | 0.5 |
| Carbomer | 1 |
| Triethanolamine | 1 |
| Water (saturated with benzyl | 90.1 |
| alcohol) | |

*Eudragit is methacrylic acid copolymer available in various grades, manufactured by Röhm GmbH & Co. KG, Darmstadt, Germany

Manufacturing process:

1. Eudragit RS 100 and butylated hydroxytoluene were dissolved in benzyl alcohol at 80-90° C.

2. The solution was cooled to room temperature.

3. Tretinoin was added to the solution of step 2 at room temperature in a dark room and mixed to get a clear solution.

4. Sodium lauryl sulphate was dissolved in water.

5. Carbomer was dispersed in the solution of step 4.

6. The dispersion of step 3 was added to the solution of step 5 and homogenized for 10 minutes at 11,000 rpm (X5200, M. Zipperer GmbH).

7. The suspension was removed from the homogeniser and stirring was continued with a mechanical stirrer.

8. Triethanolamine was added to dispersion of step 7 to form a gel having a pH of 6.5-7, after 1 g of gel was diluted to 100 ml with water.

The gel, when applied to the skin of a subject, demonstrated no signs of grittiness, was smooth to the feel and readily spreadable.

The dispersion composition formed a smooth film with a discontinuous hydrophobic phase comprising the tretinoin and a continuous hydrophilic phase. The gel was also readily washable from the skin.

### EXAMPLE 5

### Tretinoin Dispersion with Eudragit L100 as the Hydrophobic Phase Polymer

| | |
|---|---|
| Component | Amount/100 g |

Oil phase

| | |
|---|---|
| Tretinoin | 0.1 |
| EUDRAGIT L 100 | 1.2 |
| Benzyl alcohol | 6 |
| Butylated hydroxytoluene | 0.1 |

Aqueous phase

| | |
|---|---|
| Sodium lauryl sulphate | 0.5 |
| Carbomer | 1 |
| Triethanolamine | 1 |
| Water (saturated with benzyl | 90.1 |
| alcohol) | |

The composition was prepared by a process similar to that described in Example 4.

The gel when applied to the skin of a subject, demonstrated no signs of grittiness, was smooth to the feel and readily spreadable.

The dispersion composition formed a smooth film with a discontinuous hydrophobic phase comprising the tretinoin and a continuous hydrophilic phase. The gel was also readily washable from the skin.

### EXAMPLE 6

### Tretinoin Dispersion with Eudragit RSPO as the Hydrophobic Phase Polymer

| | |
|---|---|
| Component | Amount/100 g |

Oil phase

| | |
|---|---|
| Tretinoin | 0.1 |
| EUDRAGIT RS PO | 1.2 |
| Benzyl alcohol | 6 |
| Butylated hydroxytoluene | 0.1 |

Aqueous phase

| | |
|---|---|
| Sodium lauryl sulphate | 0.5 |
| Carbomer | 1 |
| Triethanolamine | 1 |
| Water (saturated with benzyl | 90.1 |
| alcohol) | |

The composition was prepared by a process similar to that described in Example 4.

The gel when applied to the skin of a subject, demonstrated no signs of grittiness, was smooth to the feel and readily spreadable.

The dispersion composition formed a smooth film with a discontinuous hydrophobic phase comprising the tretinoin and a continuous hydrophilic phase. The gel was also readily washable from the skin.

### EXAMPLE 7

### Tretinoin Dispersion with Cellulose Acetate Phthalate as the Hydrophobic Phase Polymer

| | |
|---|---|
| Component | Amount/100 g |

Oil phase

| | |
|---|---|
| Tretinoin | 0.1 |
| Cellulose acetate phthalate | 1.2 |
| Benzyl alcohol | 6 |
| Butylated hydroxytoluene | 0.1 |

Aqueous phase

| | |
|---|---|
| Sodium lauryl sulphate | 0.5 |
| Carbomer | 1 |
| Triethanolamine | 1 |
| Water (saturated with benzyl | 90.1 |
| alcohol) | |

The composition was prepared by a process similar to that described in Example 4 except that Eudragit RS100 was replaced with cellulose acetate phthalate.

The gel when applied to the skin of a subject, demonstrated no signs of grittiness, was smooth to the feel and readily spreadable.

The dispersion composition formed a smooth film with a discontinuous hydrophobic phase comprising the tretinoin and a continuous hydrophilic phase. The gel was also readily washable from the skin.

### EXAMPLE 8

### Tretinoin Dispersion with Glyceryl Monostearate as the Hydrophobic Phase Material

| Component | Amount/100 g |
|---|---|
| Tretinoin | 0.1 |
| Triacetin | 10 |
| Glyceryl monostearate | 3 |
| Carbomer | 1 |
| Tromethamine solution (20% w/w in | 0.85 |
| water) | |
| Water (saturated with benzyl alcohol) | 85.05 |

Manufacturing process:

1. Triacetin and glyceryl monostearate were heated to 60-70° C. to form a clear solution.

2. Tretinoin was added to the solution of step 1 and the temperature was maintained as in step 1 until a clear solution was achieved.

3. Carbomer was dispersed in water with stirring.

4. The solution of step 2 was added to the dispersion of step 3 and homogenized for 10 minutes at 11,000 rpm using a probe homogenizer (X5200, M. Zipperer GmbH, Staufen, Germany).

5. Tromethamine solution was added to the mixture of step 4 to form a gel.

### EXAMPLE 9

### Tretinoin Composition with Glyceryl Monostearate and Sodium Lauryl Sulphate

| Component | Amount/100 g |
|---|---|
| Tretinoin | 0.1 |
| Triacetin | 10 |
| Glyceryl monostearate | 3 |
| Sodium lauryl sulphate | 0.05 |
| Carbomer | 1 |
| Tromethamine solution (20% w/w in | 0.85 |
| water) | |
| Water (saturated with benzyl alcohol) | 85 |

Manufacturing process:

1. Triacetin and glyceryl monostearate were heated to 60-70° C. to form a clear solution.

2. Tretinoin was added to the solution of step 1 and the temperature was maintained as in step 1 until a clear solution was achieved.

3. Sodium lauryl sulphate was dissolved in water.

4. Carbomer was dispersed in the solution of step 3 accompanied by stirring.

5. The solution of step 2 was added to the dispersion of step 4 and homogenized for 10 minutes at 11,000 rpm using a probe homogenizer (X5200, M. Zipperer GmbH).

Tromethamine solution was added to mixture of step 5 and the mixture was stirred using an overhead stirrer (RQ129D, Remi Instrument Ltd, India) at 400 rpm for 10 minutes to form a gel.

The composition was easy to apply and to remove as well when tested on subjects. The gel exhibited a smooth cooling feel when applied and formed a thin film over the skin surface.

The dispersion composition formed a smooth film with a discontinuous hydrophobic phase comprising the tretinoin and a continuous hydrophilic phase. The gel was also readily washable from the skin.

### EXAMPLE 10

### Tretinoin Dispersion with Stearic Acid as the Hydrophobic Phase Material

| Component | Amount/100 g |
|---|---|
| Tretinoin | 0.1 |
| Triacetin | 1 |
| Stearic acid | 10 |
| Sodium lauryl sulphate | 0.05 |
| Butylated hydroxytoluene | 0.1 |
| Carbomer | 1 |
| Tromethamine solution (20% w/w in | 0.85 |
| water) | |
| Water (saturated with benzyl alcohol) | 86.9 |

The composition was prepared by a process similar to Example 9 except that glyceryl monostearate was replaced with stearic acid in step 1.

### EXAMPLE 11

### Tretinoin Dispersion with a Combination of a Polymer and a Lipid as the Hydrophobic Phase Material

| | |
|---|---|
| Component | Amount/100 g |

Oil phase

| | |
|---|---|
| Tretinoin | 0.1 |
| Ethyl cellulose 10 cps | 0.6 |
| Stearic acid | 0.6 |
| Butylated hydroxytoluene | 0.1 |
| Benzyl alcohol | 6 |

Aqueous phase

| | |
|---|---|
| Carbomer | 1 |
| Sodium lauryl sulphate | 0.5 |
| Triethanolamine | 1 |
| Water (saturated with benzyl alcohol) | 90.1 |

The composition was prepared by a process similar to Example 4 except that ethyl cellulose, stearic acid and butylated hydroxytoluene were dissolved in benzyl alcohol at 70-80° C. in step 1.

### EXAMPLE 12

### Tretinoin Dispersion with a Combination of Ethyl Cellulose and Glyceryl Monostearate as the Hydrophobic Phase Material

| | |
|---|---|
| Component | Amount/100 g |

Oil phase

| | |
|---|---|
| Tretinoin | 0.1 |
| Ethyl cellulose 10 cps | 0.6 |
| Glyceryl monostearate | 0.6 |
| Butylated hydroxytoluene | 0.1 |
| Benzyl alcohol | 6 |

Aqueous phase

| | |
|---|---|
| Carbomer | 1 |
| Sodium lauryl sulphate | 0.5 |
| Triethanolamine | 1 |
| Water (saturated with benzyl alcohol) | 90.1 |

The composition was prepared by a process similar to Example 4 except that ethyl cellulose, glyceryl monostearate and butylated hydroxytoluene were dissolved in benzyl alcohol at 70-80° C. in step 1.

### EXAMPLE 13

### Tretinoin Dispersion with a Combination of Ethyl Cellulose and Eudragit RS100 as the Hydrophobic Phase Material

| | |
|---|---|
| Component | Amount/100 g |

Oil phase

| | |
|---|---|
| Tretinoin | 0.1 |
| Ethyl cellulose 10 cps | 0.6 |
| Eudragit RS100 | 0.6 |
| Butylated hydroxytoluene | 0.1 |
| Benzyl alcohol | 6 |

Aqueous phase

| | |
|---|---|
| Carbomer | 1 |
| Sodium lauryl sulphate | 0.5 |
| Triethanolamine | 1 |
| Water (saturated with benzyl alcohol) | 90.1 |

The composition was prepared by a process similar to Example 4 except that ethyl cellulose, Eudragit RS100 and butylated hydroxytoluene were dissolved in benzyl alcohol at 70-80° C. in step 1.

### EXAMPLE 14

### Composition with Combination of Solvents in the Oil Phase

| | |
|---|---|
| Component | Amount/100 g |

Oil phase

| | |
|---|---|
| Tretinoin | 0.1 |
| Ethyl cellulose 10 cps | 1.2 |
| Butylated hydroxytoluene | 0.1 |
| Propylene glycol | 3 |
| Benzyl alcohol | 6 |

Aqueous phase

| | |
|---|---|
| Carbomer | 1 |
| Sodium lauryl sulphate | 0.5 |
| Triethanolamine | 1 |
| Water (saturated with benzyl alcohol) | 87.1 |

The composition was prepared by a process similar to Example 4 except that ethyl cellulose and butylated hydroxytoluene were dissolved in benzyl alcohol and propylene glycol at 70-80° C. in step 1.

### EXAMPLES 15-17

### Tretinoin Dispersion with Different Concentrations of Ethyl Cellulose as the Hydrophobic Phase Polymer

| | | | |
|---|---|---|---|
| | EXAMPLE | EXAMPLE | |
| | 15 | 16 | EXAMPLE 17 |
| | Amount/ | Amount/ | Amount/ |
| Component | 100 g | 100 g | 100 g |

Oil phase

| | | | |
|---|---|---|---|
| Tretinoin | 0.1 | 0.1 | 0.1 |
| Ethyl cellulose 10 cps | 2 | 0.8 | 1.2 |
| Benzyl alcohol | 6 | 6 | 6 |
| Butylated hydroxytoluene | 0.1 | 0.1 | 0.1 |

Aqueous phase

| | | | |
|---|---|---|---|
| Sodium lauryl sulphate | 0.5 | 0.5 | 0.5 |
| Carbomer | 1 | 1 | 1 |
| Triethanolamine | 1 | 1 | 1 |
| Water (saturated with | 89.3 | 90.5 | 90.1 |
| benzyl alcohol) | | | |

The compositions were prepared by a process similar to Example 4 except that ethyl cellulose and butylated hydroxytoluene were dissolved in benzyl alcohol and propylene glycol at 70-80° C. in step 1.

### EXAMPLE 18

### In-Vitro Drug Release of Tretinoin from the Composition of Example 17

The dispersion composition prepared in Example 17 was subjected to a drug release study using VanKel dissolution cell. It contains an enhancer cell assembly, a dissolution flask and a paddle. The enhancer cell assembly is a Teflon assembly with adjustable volume and a screw cap to retain the membrane or skin.

About 200 mg of composition was placed in the enhancer cell and a Millipore 0.45 μm membrane filter was placed. The washer was placed over the membrane and retaining ring was screwed on. The enhancer cell was placed in the dissolution flask containing 200 ml of dissolution medium; 0.1 N sodium hydroxide containing 0.5% hydroxypropyl betacyclodextrin and 4% benzyl alcohol was used as dissolution medium. The temperature of the dissolution flask was maintained at 34±0.5° C. and the medium was stirred at 100 rpm. Samples were analyzed for tretinoin by HPLC.

| Time | |
|---|---|
| (hours) | Drug released, % |
| 1 | 46.82 |
| 2 | 54.28 |
| 4 | 62.94 |
| 6 | 68.64 |
| 8 | 69.36 |

### EXAMPLE 19

### Comparative Irritancy Study of a Dispersion Composition of the Invention Comprising a Tretinoin Irritant and a Commercial Product

A comparative study of the composition of Example 17 and the commercial product RETIN-A MICRO™ (tretinoin gel) microsphere, a product of the OrthoNeutrogena division of Ortho-McNeil Pharmaceuticals, Inc. containing tretinoin (0.1% w/w), was performed to evaluate the intensity of surface irritation including stinging on application to the skin of human volunteers. A total of 31 volunteer subjects in the age range of 20-38 years participated in the study, comprising 27 males and 4 females. About 0.1 g portions of the compositions were spread onto 2 cm×4 cm areas on different forearms, without rubbing, and left undisturbed for 10 minutes. Subject observations were recorded at the time of application and at 1, 2, 5, and 10 minutes thereafter. The irritation intensity/stinging was recorded on a 0-4 scale where 0 indicated no irritation, 1 indicated slight irritation, 2 indicated mild irritation, 3 indicated significant irritation, and 4 indicated severe irritation.

Sixteen subjects reported irritation from the commercial product application, with scores of 1, 2, 3, or 4, while six subjects reported irritation from application of the Example 17 composition, with scores of 1 or 2. The differences in the composition of Example 17 and the commercial product of tretinoin appear to be significant in that the former caused less irritation, and the onset of irritation was delayed and persisted for a shorter time.

### EXAMPLE 20

### Finasteride Dispersion with Ethyl Cellulose as the Hydrophobic Phase Polymer

| | |
|---|---|
| Component | Amount/100 g |

Oil phase

| | |
|---|---|
| Finasteride | 0.1 |
| Ethyl cellulose 10 cps | 1.2 |
| Benzyl alcohol | 6 |
| Butylated hydroxytoluene | 0.1 |

Aqueous phase

| | |
|---|---|
| Sodium lauryl sulphate | 0.5 |
| Carbomer | 1 |
| Triethanolamine | 1 |
| Water (saturated with benzyl | 90.1 |
| alcohol) | |

The composition was prepared by a process similar to that of Example 15 except that tretinoin was replaced with finasteride.

### EXAMPLE 21

### Diclofenac Sodium Dispersion with Ethyl Cellulose as the Hydrophobic Phase Polymer

| | |
|---|---|
| Component | Amount/100 g |

Oil phase

| | |
|---|---|
| Diclofenac sodium | 0.1 |
| Ethyl cellulose 10 cps | 1.2 |
| Benzyl alcohol | 6 |
| Butylated hydroxytoluene | 0.1 |

Aqueous phase

| | |
|---|---|
| Sodium lauryl sulphate | 0.5 |
| Carbomer | 1 |
| Triethanolamine | 1 |
| Water (saturated with benzyl | 90.1 |
| alcohol) | |

The composition was prepared by a process similar to that of Example 15 except that tretinoin was replaced with diclofenac sodium.

This example demonstrates that the dispersion composition can also be prepared for other classes of compounds such as anti-inflammatory agents which are used for local application.

### EXAMPLE 22

### Tretinoin Dispersion with Ethyl Cellulose as the Hydrophobic Phase Polymer and Glycerol as Emollient

| | |
|---|---|
| Component | Amount/100 g |

Oil phase

| | |
|---|---|
| Tretinoin | 0.1 |
| Ethyl cellulose 10 cps | 1.2 |
| Butylated hydroxytoluene | 0.1 |
| Glycerol | 3 |
| Benzyl alcohol | 6 |

Aqueous phase

| | |
|---|---|
| Carbomer | 1 |
| Sodium lauryl sulphate | 0.5 |
| Triethanolamine | 1 |
| Water (saturated with benzyl alcohol) | 87.1 |

Manufacturing process:

1. Ethyl cellulose and butylated hydroxytoluene were dissolved in benzyl alcohol at 80-90° C., glycerol was added.

2. The solution was cooled to room temperature.

3. Tretinoin was added to the solution of step 2 at room temperature in a dark room and mixed to get a clear solution.

4. Sodium lauryl sulphate was dissolved in water.

5. Carbomer was dispersed in the solution of step 4.

6. The dispersion of step 3 was added to the solution of step 5 and homogenized for 10 minutes at 11,000 rpm (X5200, M. Zipperer GmbH).

7. The suspension was removed from the homogeniser and stirring was continued with a mechanical stirrer.

8. Triethanolamine was added to dispersion of step 7 to form a gel having a pH of 6.5-7, after 1 g of gel was diluted to 100 ml with water.

The gel, when applied to the skin of a subject, demonstrated no signs of grittiness, was smooth to the feel and readily spreadable.

The dispersion composition formed a smooth film with a discontinuous hydrophobic phase comprising the tretinoin and a continuous hydrophilic phase. The gel was also readily washable from the skin.

This example demonstrates that the dispersion composition can also be prepared using bioinactive agents which would find use in cosmetic applications. Similar compositions could also be prepared using other bioinactive agents for cosmetic applications.

### EXAMPLE 23

### Tretinoin and Finasteride Dispersion with Ethyl Cellulose as the Hydrophobic Phase Polymer

| | |
|---|---|
| Component | Amount/100 g |

Oil phase I

| | |
|---|---|
| Tretinoin | 0.1 |
| Ethyl cellulose 10 cps | 1.2 |
| Butylated hydroxytoluene | 0.1 |
| Glycerol | 3 |
| Benzyl alcohol | 6 |

Oil phase II

| | |
|---|---|
| Finasteride | 0.1 |
| Ethyl cellulose 10 cps | 1.2 |
| Butylated hydroxytoluene | 0.1 |
| Glycerol | 3 |
| Benzyl alcohol | 6 |

Aqueous phase

| | |
|---|---|
| Carbomer | 1 |
| Sodium lauryl sulphate | 0.5 |
| Triethanolamine | 1 |
| Water (saturated with benzyl alcohol) | 82.7 |

Manufacturing process:

Oil phase I

1. Ethyl cellulose 10 cps and butylated hydroxytoluene were dissolved in benzyl alcohol at 80-90° C.

2. The solution was cooled to room temperature.

3. Tretinoin was added to the solution of step 2 at room temperature in a dark room and mixed to get a clear solution.

Oil phase II

4. Ethyl cellulose 10 cps and butylated hydroxytoluene were dissolved in benzyl alcohol at 80-90° C.

5. The solution was cooled to room temperature.

6. Finasteride was added to the solution of step 5 at room temperature in a dark room and mixed to get a clear solution.

Aqueous phase

7. Sodium lauryl sulphate was dissolved in water (previously saturated with benzyl alcohol).

8. Carbomer was dispersed in the solution of step 7.

9. The solutions of step 3 and step 6 were added to the dispersion of step 8 and homogenized for 10 minutes at 11,000 rpm (X5200, M. Zipperer GmbH).

10. The dispersion was removed from the homogeniser and stirring was continued with a mechanical stirrer.

11. Triethanolamine was added to dispersion of step 10 to form a gel.

### EXAMPLE 24

### Tretinoin Dispersion with Ethyl Cellulose as the Hydrophobic Phase Polymer and a Combination of Solvents in the Aqueous Phase

| | |
|---|---|
| Component | Amount/100 g |

Oil phase

| | |
|---|---|
| Tretinoin | 0.1 |
| Ethyl cellulose 10 cps | 1.2 |
| Butylated hydroxytoluene | 0.1 |
| Benzyl alcohol | 6 |

Aqueous phase

| | |
|---|---|
| Carbomer | 1 |
| Sodium lauryl sulphate | 0.5 |
| Triethanolamine | 1 |
| Propylene glycol | 5 |
| Water (saturated with benzyl alcohol) | 85.1 |

The composition was prepared by a process similar to Example 4, except that ethyl cellulose and butylated hydroxytoluene were dissolved in benzyl alcohol in step 1 and sodium lauryl sulphate was dissolved in a mixture of water and propylene glycol in step 4.

### EXAMPLE 25

### Tretinoin Dispersion with Ethyl Cellulose as a Hydrophobic Phase Polymer and a Combination of Solvents in the Aqueous Phase

| | |
|---|---|
| Component | Amount/100 g |

Oil phase

| | |
|---|---|
| Tretinoin | 0.1 |
| Ethyl cellulose 10 cps | 1.2 |
| Butylated hydroxytoluene | 0.1 |
| Benzyl alcohol | 6 |

Aqueous phase

| | |
|---|---|
| Carbomer | 1 |
| Sodium lauryl sulphate | 0.5 |
| Triethanolamine | 1 |
| Glycerol | 5 |
| Water (saturated with benzyl alcohol) | 85.1 |

The composition was prepared by a process similar to Example 4, except that ethyl cellulose and butylated hydroxytoluene were dissolved in benzyl alcohol in step 1, and sodium lauryl sulphate was dissolved in a mixture of water and glycerol in step 4.

1. A composition for topical application comprising:
a dispersed phase comprising an active agent and a polymer or lipid; and
a gelled continuous phase comprising a viscosity modifier and a solvent.

2. The composition of claim 1, wherein a dispersed phase comprises a nonaqueous solvent.

3. The composition of claim 1, wherein a gelled continuous phase comprises an aqueous solvent.

4. The composition of claim 1, wherein a dispersed phase comprises a nonaqueous solvent and a gelled continuous phase comprises an aqueous solvent.

5. The composition of claim 1, wherein a dispersed phase comprises a nonaqueous solvent and a polymer, a lipid, or both a polymer and a lipid.

6. The composition of claim 1, wherein a viscosity modifier comprises a hydrophilic substance that enhances viscosity.

7. The composition of claim 1, wherein a viscosity modifier comprises a crosslinked polyacrylic acid polymer.

8. The composition of claim 1, wherein an active agent comprises a bioactive agent.

9. The composition of claim 1, wherein an active agent comprises at least one of a peptide drug, a protein drug, a desensitizing agent, an antigen, a vaccine, an anti-infective, an antibiotic, an antifungal, an antiacne drug, a vitamins or vitamin analog, a retinoid, an antileprotic, an antimicrobial, a antineoplastic, an antitumor drug, an antiallergenic, a steroidal anti-inflammatory agent, an anesthetic, a decongestant, a miotic, an anticholinergic, a sympathomimetic drug, a sedative, a hypnotic, an antipsychotic, a psychic energizer, a tranquilizer, an androgenic steroid, an estrogen, a progestational agent, a humoral agent, a prostaglandin, an analgesic, an antispasmodic, an antimalarial, an antihistamine, a cardioactive agent, a non-steroidal anti-inflammatory agent, an antiparkinsonian agent, an antihypertensive agent, a beta-adrenergic blocking agent, a nutritional agent, an antiviral, a DNA fragment, a nucleic acid, genetic material, an oligonucleotide, and a radioisotope.

10. The composition of claim 1, comprising more than one active agent.

11. The composition of claim 1, wherein an active agent comprises at least one bioactive agent and at least one bioinactive agent.

12. The composition of claim 1, wherein a film formed after solvent evaporation releases active agent during a prolonged time.

13. The composition of claim 1, wherein an active agent comprises a retinoid.

14. A process for preparing a composition for topical application, comprising:
combining components comprising an active agent and a polymer or lipid to form a dispersible phase;
combining components comprising a viscosity modifier and a solvent, to form a gel-forming composition; and
dispersing a dispersible phase in a gel-forming composition to form a dispersion having a gel continuous phase.

15. The process of claim 14, wherein an active agent comprises a bioactive agent.

16. The process of claim 14, wherein a solvent is aqueous.

17. The process of claim 14, wherein a dispersible phase is hydrophobic and a gel is hydrophilic.

18. The process of claim 14, wherein a dispersible phase comprises a hydrophobic solvent.

19. The process of claim 14, wherein an active agent comprises a bioactive agent and a bioinactive agent.

20. The process of claim 14, wherein an active agent comprises a retinoid.

21. The process of claim 14, wherein a gel is formed after dispersing a dispersible phase in components comprising a viscosity modifier and a solvent.

22. A process for preparing a composition for topical application, comprising:
dissolving tretinoin in a dispersible composition comprising a polymer or an oil;
combining gel-forming composition components comprising water and a polymer; and
dispersing a dispersible composition in a gel-forming composition.
